(19) United States
(12) Patent Application Publication
(10) Pub. No.: US 2025/0268802 A1
(43) Pub. Date: Aug. 28, 2025

(54) TEMPERATURE STABLE EMULSIONS

(71) Applicant: BEIERSDORF AG, Hamburg (DE)

(72) Inventors: Wei TANG, Hamburg (DE); Kerstin FRANCK, Quickborn (DE); Yang XU, Hamburg (DE)

(21) Appl. No.: 18/857,689

(22) PCT Filed: Apr. 17, 2023
(86) PCT No.: PCT/EP2023/059901

§ 371 Date: Oct. 17, 2024

(30) Foreign Application Priority Data
May 12, 2022 (DE) 10 2022 204 660.9

(51) Int. Cl. A61K 8/02; A61K 8/06; A61K 8/34; A61K 8/37; A61K 8/49; A61K 8/73; A61Q 19/10

(52) U.S. Cl. CPC A61K 8/0295; A61K 8/062; A61K 8/342; A61K 8/375; A61K 8/4973; A61K 8/73; A61K 8/732; A61Q 19/10

(57) ABSTRACT

The invention relates to a cosmetic emulsion.

Cosmetic products generally not only contribute to a beautiful and attractive appearance, but their effects make a crucial contribution to increased self-esteem and people's wellbeing. Accordingly, a wide variety of cosmetic products are used for the daily cleansing and care of human skin.

Skin care products generally consist of emulsions. Emulsions are generally understood to mean heterogeneous systems composed of two liquids that are immiscible, or of only limited miscibility, with each other, which are typically referred to as phases and in which one of the two liquids is dispersed in the other liquid in the form of very fine droplets. Externally and viewed with the naked eye, emulsions appear homogeneous.

If the two liquids are water and oil and oil droplets are present finely distributed in water, it is an oil-in-water emulsion (O/W emulsion, for example milk). The basic character of an O/W emulsion is influenced by the water. The reverse principle applies to a water-in-oil emulsion (W/O emulsion, for example butter), the basic character being determined in this case by the oil.

To stabilize and thicken O/W emulsions, acrylate-based polymers are usually incorporated into these formulations. Acrylate-based polymers are polymers that are obtained from homo- or copolymerization with acrylic and/or methacrylic acid. Examples of these include Carbomer or Acrylates Copolymer.

Emulsions with acrylates are known from documents including DE 10148825 A1, DE 19938756 A1 and DE 29924371 U1.

Formulations with acrylate-based polymers have a whole range of advantages. For example, the formulations often feature low stickiness. Furthermore, the formulations are well protected against formation of flocs and/or clumps when stored for relatively long periods for example for 30 days at −10° C. If the temperature of the formulation is increased back to 20° C. after storage for 30 days at −10° C., this formulation is described as soft and smooth.

However, the use of these acrylate-based polymers is increasingly being subject to criticism since their biodegradability has not been entirely clarified.

A disadvantage of the prior art is that it is almost impossible to combine low stickiness and temperature stability against flocs or clump formation at −10° C. for several months without the use of acrylate-based polymers. It is known to those skilled in the art to use short-chain diols, which results in better temperature stability, but at the same time increases the stickiness of the formula. The use of individual biopolymers or different combinations often results in inadequate temperature stabilities or high stickiness.

Surprisingly, the present invention was able to address the problems of the prior art.

Subject matter of the present invention is an acrylate-free cosmetic emulsion comprising
a) an emulsifier system which forms a liquid-crystalline network in the emulsion,
b) hydroxypropyl starch phosphate and/or Distarch Phosphate
c) Gellan Gum.

By definition, acrylate-free means that no polymers obtained from homo-or copolymerization with acrylic and/or methacrylic acid are present.

By definition, fatty alcohols have a C10 to C20 carbon chain. Correspondingly, the alcohol radicals of the fatty alcohol glucosides also have such a carbon chain.

By definition, the presence of the emulsifier system results in the formation of a liquid-crystalline network in the formulation. A liquid-crystalline network is characterized and defined in that liquid-crystalline structures are formed. By definition, the emulsifier system must contribute to the formation of liquid-crystalline structures.

Subject matter of the invention also is the use of the emulsion of the invention for providing a cosmetic product.

This is preferably an oil-in-water (O/W) emulsion.

If percentages by weight (% by weight) are stated below without reference to a certain composition or specific mixture, then these figures always relate to the total weight of the cosmetic emulsion. If ratios of components/substances/substance groups are disclosed below, then these ratios relate to weight ratios of the components/substances/substance groups mentioned.

If weight percentage ranges for the constituents of the cosmetic emulsion are stated below, then the disclosure of the present application also encompasses all individual values in steps of 0.1% by weight within these weight percentage ranges.

Within the scope of the present disclosure, the phrases “according to the invention”, “advantageous according to the invention”, “advantageous in the context of the present invention” etc. always relate to both the preparation according to the invention and the use according to the invention.

Unless stated otherwise, all experiments were carried out under standard conditions. The expression “standard conditions” means 20° C., 1013 hPa and a relative humidity of 50%.

If the term skin is used, then this preferably relates to human skin.

If viscosity values are stated in this disclosure, then all values relate to a measurement at 25° C. in a 150 ml wide-neck bottle (VWR No.: 807-001) by means of a Rheomat R 123 from proRheo. The Rheomat R 123 from proRheo GmbH is a rotational viscometer, i.e. a measuring body rotates in the substance to be measured. The force required to rotate the measuring body in the sample at a defined speed is measured. The viscosity is calculated from this torque, the speed of the measuring body and the geometric dimensions of the measuring system used. The measuring body used is the No. 2 measuring body (Article No. 200 0192), speed range 62.5 min⁻¹.

According to the invention, the emulsion has an emulsifier system which forms a liquid-crystalline network in the emulsion.

The presence of such a network is correspondingly a feature of the emulsion according to the invention. It is advantageously a liquid-crystalline gel network.

It has surprisingly been found that the gel network specifically formed in this invention, in combination with the further components of the invention, makes it possible to provide acrylate-free emulsions which exhibit less clump formation even when stored at −10° C.

The emulsifier system according to the invention advantageously comprises at least one fatty alcohol glucoside. Advantageously, the fatty alcohol glucoside is selected from the group having the INCI names Arachidyl Glucoside; C10-16 Alkyl Glucoside; C12-18 Alkyl Glucoside; C12-20 Alkyl Glucoside; Coco-glucoside; C9-11 Alkyl Glucoside; Caprylyl/Capryl Glucoside; Caprylyl Glucoside; Cetearyl Glucoside; Decyl Glucoside; Isostearyl Glucoside; Lauryl Glucoside; Myristyl Glucoside and/or Undecyl Glucoside. Preference is given to Cetearyl Glucoside, Caprylyl Glucoside, Caprylyl/Capryl Glucoside, Lauryl Glucoside and Myristyl Glucoside. Cetearyl Glucoside is especially preferred.

Advantageously, the proportion by weight of the entirety of the fatty alcohol glucosides in the emulsion is from 0.1% to 5.0% by weight, preferably from 0.5% to 3.5% by weight, preferably 0.6% to 2.5% by weight and especially preferably from 0.9% to 2.2% by weight, in each case based on the total weight of the emulsion.

It is also advantageous if the total proportion by weight of Arachidyl Glucoside; C10-16 Alkyl Glucoside; C12-18 Alkyl Glucoside; C12-20 Alkyl Glucoside; Coco-glucoside; C9-11 Alkyl Glucoside; Caprylyl/Capryl Glucoside; Caprylyl Glucoside; Cetearyl Glucoside; Decyl Glucoside; Isostearyl Glucoside; Lauryl Glucoside; Myristyl Glucoside and/or Undecyl Glucoside is from 0.1% to 5.0% by weight, preferably from 0.5% to 3.5% by weight, preferably 0.6% to 2.5% by weight and especially preferably from 0.9% to 2.2% by weight, in each case based on the total weight of the emulsion.

It is especially advantageous if the proportion of Cetearyl Glucoside, Caprylyl Glucoside, Caprylyl/Capryl Glucoside, Lauryl Glucoside and/or Myristyl Glucoside is from 0.1% to 5.0% by weight, preferably from 0.5% to 3.5% by weight, preferably 0.6% to 2.5% by weight and especially preferably from 0.9% to 2.2% by weight, in each case based on the total weight of the emulsion.

It is especially advantageous if the proportion of Cetearyl Glucoside is from 0.1% to 5.0% by weight, preferably from 0.5% to 3.5% by weight, preferably 0.6% to 2.5% by weight and especially preferably from 0.9% to 2.2% by weight, in each case based on the total weight of the emulsion.

Furthermore, it is advantageous if the emulsifier system comprises Sorbitan Stearate. If Sorbitan Stearate is present, the total proportion of Sorbitan Stearate is advantageously from 0.1% to 1.5% by weight, preferably 0.15% to 1.0% by weight and in particular 0.2% to 0.8% by weight, based on the total weight of the emulsion.

Furthermore, it is advantageous if the emulsifier system comprises Disodium Cetearyl Sulfosuccinate. If Disodium Cetearyl Sulfosuccinate is present, the total proportion of Disodium Cetearyl Sulfosuccinate is advantageously from 0.01% to 0.5% by weight, preferably 0.02% to 0.2% by weight and in particular 0.04% to 0.10% by weight, based on the total weight of the emulsion.

Furthermore, it is advantageous if the emulsifier system comprises Glyceryl Stearate. If Glyceryl Stearate is present, the total proportion of Glyceryl Stearate is advantageously from 0.1% to 1.8% by weight, preferably 0.5% to 1.5% by weight and in particular 0.7% to 1.2% by weight, based on the total weight of the emulsion.

Furthermore, the emulsifier system advantageously additionally comprises myristyl alcohol. Advantageously, the proportion of the myristyl alcohol is from 0.2% to 6.5% by weight, preferably 0.5% to 6.0% by weight, preferably 1.5% to 5.0% by weight, preferably 2.5% to 4.5% by weight and especially preferably 3.0% to 4.0% by weight, in each case based on the total weight of the emulsion.

Liquid-crystalline structures in emulsions can be visualized by means of light microscopy with polarized light. Reference is made to the publication “Study on the Formation and Properties of Liquid Crystal Emulsion in Cosmetic”, Wanping Zhang*, Lingyan Liu, School of Perfume and Aroma technology, Shanghai Institute of Technology, Shanghai, China. Journal of Cosmetics, Dermatological Sciences and Applications, 2013.

Advantageously, the liquid-crystalline structures in the emulsion of the invention are present in globular structure. This is identifiable by the appearance of the typical Maltese crosses in light microscopy images with polarized light.

The emulsion of the invention also comprises at least Hydroxypropyl Starch Phosphate and/or Distarch Phosphate. Advantageously, the total proportion of Hydroxypropyl Starch Phosphate and/or Distarch Phosphate is from 0.1% to 3.0% by weight, preferably 0.2% to 1.5% by weight and especially preferably 0.25% to 0.9% by weight, based on the total weight of the emulsion.

Furthermore, the emulsion of the invention comprises Gellan Gum. Advantageously, the proportion of Gellan Gum is from 0.01% by weight to 0.5% by weight, preferably 0.03% by weight to 0.3% by weight and especially preferably 0.04% to 0.2% by weight, based on the total weight of the emulsion.

Furthermore, it is advantageous if Xanthan Gum is additionally present. If Xanthan Gum is present, it is preferred if the proportion of Xanthan Gum is from 0.01% by weight to 0.5% by weight, preferably 0.03% by weight to 0.3% by weight and especially preferably 0.04% to 0.2% by weight, based on the total weight of the emulsion.

Furthermore, it is advantageous if further polysaccharides are additionally present. These further polysaccharides may advantageously be selected from further polysaccharides from the groups of the polysaccharide gums, the starches and/or celluloses selected.

Among the polysaccharide gums, use is preferably made in particular of the substances known under the INCI names Guar Gum, Sclerotium Gum and/or Tara Gum.

Among the starches, use is preferably made of the substances known under the INCI names Zea Mays Starch, Sodium Hydroxypropyl Starch Phosphate, Sodium Carboxymethyl Starch, Tapioca Starch and/or Potato Starch Modified.

Among the celluloses, use is preferably made of the substances known under the INCI names Ethylcellulose, Microcrystalline Cellulose, Cellulose Gum, Hydroxyethylcellulose, Carboxymethyl Hydroxyethylcellulose, Hydroxyethyl Ethylcellulose, Hydroxymethyl Cellulose, Hydroxypropylcellulose, Hydroxypropyl Methylcellulose and/or Hydroxyethylcellulose.

Advantageously, the proportion of the further polysaccharides in the emulsion is from 0.01% to 4% by weight, preferably 0.3% to 2.5% by weight, preferably from 0.5% to 1.5% by weight and especially preferably from 0.6% to 1.0% by weight, based on the total weight of the emulsion.

Advantageously, the proportion of the further polysaccharide gums in the emulsion is from 0.01% to 4% by weight, preferably 0.3% to 2.5% by weight, preferably from 0.5% to 1.5% by weight and especially preferably from 0.6% to 1.0% by weight, based on the total weight of the emulsion.

Advantageously, the proportion of the further starches in the emulsion is from 0.01% to 4% by weight, preferably 0.3% to 2.5% by weight, preferably from 0.5% to 1.5% by weight and especially preferably from 0.6% to 1.0% by weight, based on the total weight of the emulsion.

Advantageously, the proportion of the celluloses in the emulsion is from 0.01% to 4% by weight, preferably 0.3% to 2.5% by weight, preferably from 0.5% to 1.5% by weight and especially preferably from 0.6% to 1.0% by weight, based on the total weight of the emulsion.

Advantageously, the total proportion of Ethylcellulose, Microcrystalline Cellulose, Cellulose Gum, Hydroxyethylcellulose, Carboxymethyl Hydroxyethylcellulose, Hydroxyethyl Ethylcellulose, Hydroxymethyl Cellulose, Hydroxypropylcellulose, Hydroxypropyl Methylcellulose and/or Hydroxyethylcellulose in the emulsion is from 0.01% to 4% by weight, preferably 0.3% to 2.5% by weight, preferably from 0.5% to 1.5% by weight and especially preferably from 0.6% to 1.0% by weight, based on the total weight of the emulsion.

It is customary in the prior art to add short-chain diols having 3 to 5 carbon atoms, such as butylene glycol or propane-1,2-diol, to emulsions in order to prevent flocculation/clump formation of formula constituents at −10° C. Surprisingly, it has now been found that such freezing stability is obtained even in the absence of these substances. It can be assumed that the polymers according to the invention, in interaction with the liquid-crystalline structures formed, contribute to the stabilization. Furthermore, it has surprisingly been found that formulations without short-chain diols having 3 to 5 carbon atoms have significantly reduced stickiness. Low stickiness is particularly a problem for acrylate-free formulations.

Consequently, it is preferred according to the invention if the proportion of short-chain diols having 3 to 5 carbon atoms amounts to less than 0.5% by weight, preferably less than 0.25% by weight, preferably less than 0.1% by weight and especially preferably 0.0% by weight, based on the total weight of the emulsion. Accordingly, it is preferred if no short-chain diols having 3 to 5 carbon atoms are present.

Furthermore, the emulsion advantageously comprises at least one wax component having a melting point of less than 43° C.

Advantageously, at least one wax component having a melting point in the range from 25° C. to 43° C. is present. The presence of such low-melting wax components ensures the melting of the preparation on human skin. Surprisingly, it was simultaneously possible to approximately maintain the consistency, in particular at temperatures around 45° C. In addition, it was surprisingly possible to prevent phase separations at 45° C.

Fatty alcohols and fatty acids having alkyl chains with 10 to 20 carbon atoms by definition are not considered to be wax components.

Advantageously, the total content of the wax components having a melting point in the range from 25° C. to 43° C. is from 0.5% to 5% by weight, preferably 1% to 4.5% by weight and especially preferably 1.5% to 3.5% by weight, based on the total weight of the emulsion.

Preferred wax components in the stated melting range are Myristyl Myristate, Hydrogenated Coco-Glycerides and/or Cetyl Ricinoleate.

Advantageously, the total content of Myristyl Myristate, Hydrogenated Coco-Glycerides and/or Cetyl Ricinoleate is from 0.5% to 5% by weight, preferably 1.5% to 4.5% by weight and especially preferably 2.2% to 3.5% by weight, based on the total weight of the emulsion.

If Myristyl Myristate is present, it is advantageous if the proportion of Myristyl Myristate is from 0.5% to 5.5% by weight, preferably 1.0% to 4.5% by weight and especially preferably from 1.4% to 3.0% by weight, based on the total weight of the emulsion. If Hydrogenated Coco-Glycerides is present, it is preferred if the proportion of Hydrogenated Coco-Glycerides is from 0.1% to 3.0% by weight, preferably 0.3% to 2.0% by weight and preferably 0.5% to 1.5% by weight, based on the total weight of the emulsion. If Cetyl Ricinoleate is present, it is preferred if the proportion of Cetyl Ricinoleate is from 0.01% to 3.0% by weight, preferably 0.05% to 1.0% by weight and preferably 0.1% to 0.5% by weight, based on the total weight of the emulsion.

Furthermore, it is advantageous according to the invention if high-melting waxes having a melting point of more than 43° C. are not present, or the proportions thereof are less than 0.5% by weight of the emulsion. Surprisingly, it was possible to provide stable formulations at 45° C., without the need to incorporate high-melting wax components. It is assumed that the formation of the liquid-crystalline structures at 45° C. enables the stability of the formulation to prevent phase separations to maintain the consistency.

Furthermore, it is advantageous if the emulsion comprises one or more oil components that are liquid under standard conditions. In general, it is preferred if the proportion of the oil components that are liquid under standard conditions is from 0.2% to 5.0% by weight, preferably 0.5% to 4.0% by weight and especially preferably 1.5% to 2.5% by weight, based on the total weight of the emulsion.

Oils that are advantageously present are selected from natural oils. Natural oils that are advantageously present are selected from the group of Persea Gratissima Oil, Orbignya Oleifera Seed Oil, Argania Spinosa Kernel Oil, Prunus Armeniaca Kernel Oil, Simmondsia Chinensis Seed Oil, Butyrospermum Parkii Butter, Cocos Nucifera Oil, Silybum Marianum Seed Oil, Oenothera Biennis Oil, Olea Europaea Fruit Oil, Helianthus Annuus Seed Oil, Vitis Vinifera Seed Oil, Cannabis Sativa Seed Oil, Vegetable Oil, Gossypium Herbaceum Seed Oil, Arctium Lappa Seed Oil, Macadamia Ternifolia Seed Oil, Macadamia Integrifolia Seed Oil, Zea Mays Germ Oil, Prunus Amygdalus Dulcis Oil, Ricinus Communis Seed Oil, Brassica Campestris Seed Oil and/or Glycine Soja Oil. If natural oils are present, the total content of these oils is advantageously from 0.01% to 3% by weight, preferably 0.1% to 1.0% by weight and especially preferably from 0.2% to 0.7% by weight, based on the total weight of the emulsion.

Furthermore, it is advantageous if one or more esters of coconut fatty alcohol are present. Use is preferably made here in particular of Coco-Caprylate/Caprate. If an ester of a coconut fatty alcohol is present, the proportion of this ester is advantageously from 0.2% to 4% by weight, preferably 0.4% to 2.5% by weight and especially preferably from 0.8% to 1.5% by weight, based on the total weight of the emulsion. If Coco-Caprylate/Caprate is present, the proportion of Coco-Caprylate/Caprate is advantageously from 0.2% to 4% by weight, preferably 0.4% to 2.5% by weight and especially preferably from 0.8% to 1.5% by weight, based on the total weight of the emulsion.

Furthermore, it is preferred if one or more oils that are triglycerides of 3 identical fatty acids are present. Triisostearin is especially preferred. If triglycerides of 3 identical fatty acids are present, the proportion of these triglycerides is advantageously from 0.05% to 2% by weight, preferably 0.08% to 1.0% by weight and especially preferably from 0.1% to 0.5% by weight, based on the total weight of the emulsion. If Triisostearin is present, the proportion of Triisostearin is advantageously from 0.05% to 2% by weight, preferably 0.08% to 1.0% by weight and especially preferably from 0.1% to 0.5% by weight, based on the total weight of the emulsion.

Additionally, it is advantageous if one or more fatty alcohols having 16 to 24 carbon atoms are present, with the total proportion of these fatty alcohols being from 0.01% to 1.5% by weight, preferably from 0.1% to 0.3% by weight, based on the total weight of the emulsion. In particular, cetearyl alcohol is present at a proportion of 0.01% to 1.5% by weight, preferably from 0.1% to 0.3% by weight, based on the total weight of emulsion.

Furthermore, it has been shown to be advantageous if one or more fatty acids having 14 to 24 carbon atoms are present in the emulsion, with the proportion thereof advantageously being from 0.2% to 5.0% by weight, preferably from 1.5% by weight to 4% by weight and especially preferably from 1.8% to 3.0% by weight, based on the total weight of the emulsion.

The emulsion according to the invention is also preferably characterized in that it comprises glycerol at a proportion of 0.5% by weight to 15% by weight, preferably 3.0% to 7% by weight, based on the total weight of the emulsion.

Furthermore, it is also advantageous in the context of the present invention if the emulsion comprises phenoxyethanol, dehydroacetic acid, benzyl alcohol and/or ethylhexylglycerin.

If the emulsion comprises benzyl alcohol, it is preferred if the proportion of benzyl alcohol is from 0.05% by weight to 0.5% by weight, based on the total weight of the emulsion.

If the emulsion comprises phenoxyethanol, it is preferred if the total proportion of phenoxyethanol is from 0.1% by weight to 1.2% by weight, based on the total weight of the emulsion.

If the emulsion comprises ethylhexylglycerin, it is preferred if the proportion of ethylhexylglycerin is from 0.1% by weight to 1.0% by weight, based on the total weight of the emulsion.

Moreover, it is advantageous if embodiments of the invention are characterized in that they comprise ethanol. If ethanol is present in the emulsion, the proportion of ethanol is preferably from 0.5% by weight to 10% by weight, preferably 1.5% by weight to 5.0% by weight, based on the total weight of the emulsion.

Last but not least, embodiments that are advantageous according to the invention are characterized in that the emulsion comprises one or more active ingredients selected from the following group of compounds: glycyrrhetinic acid, urea, arctiin, folic acid, coenzyme Q10 (ubiquinone), alpha-glucosylrutin, carnitine, carnosine, caffeine, natural and/or synthetic isoflavonoids, glycerylglucose, creatine, creatinine, taurine, tocopherol, tocopherol acetate, vitamin C, vitamin C phosphate, vitamin C palmitate, niacinamide, vitamin A palmitate, panthenol, licochalcone A, rucinol, N-[(2,4-dihydroxyphenyl)thiazol-2-yl]isobutyramide, honokiol and magnolol (also as constituent of magnolia extracts), hyaluronic acid and/or silymarin (milk thistle extract).

Emulsions that are advantageous according to the invention are also characterized in that they comprise water at a proportion of 60% by weight to 95% by weight and preferably from 70% by weight to 90% by weight, based on the total weight of the emulsion.

Furthermore, emulsions that are advantageous according to the invention have a viscosity 24 h after preparation of 8000 mPa·s to 30 000 mPa·s. If viscosity is referred to in this disclosure, then all values relate to a measurement at 25° C. in a 150 ml rolled rim glass bottle by means of a Rheomat R 123 from proRheo. The Rheomat R 123 from proRheo GmbH is a rotational viscometer, i.e. a measuring body rotates in the substance to be measured. The force required to rotate the measuring body in the sample at a defined speed is measured. The viscosity is calculated from this torque, the speed of the measuring body and the geometric dimensions of the measuring system used. The measuring body used is the No. 2 measuring body (Article No. 200 0192), speed range 62.5 min⁻¹. All measurements for viscosity are always performed 24 h after preparation of the emulsion.

### Comparative Experiments and Examples

The examples below are intended to illustrate the present invention without limiting it. Unless stated otherwise, all amounts, proportions and percentages are based on the weight and the total amount or on the total weight of the preparations.

The table below with Ex. 1 to Ex. 11 shows different formulations. Ex. 1 is a non-inventive example that represents a customary formulation having the acrylate-based polymer Carbomer. The formulation of Ex. 1 was examined after storage for 30 days at −10° and subsequent thawing to 20° C. The formulation has low stickiness. In addition, after thawing it exhibits low clump formation and is accordingly uniform, smooth and soft. The formulation does not have any liquid-crystalline structures.

Ex. 2 is a non-inventive example in which the addition of polymers was dispensed with. The same storage and thawing process as in Ex. 1 was applied. The formulation features low stickiness, however significantly increased clumping is observed after thawing.

Examples 3 to 7 are also non-inventive reference examples, which have a tendency toward increased clump formation after thawing. Various polymers are used here.

Examples 8 to 10 are example formulations according to the invention. These have globular liquid-crystalline structures, which were demonstrated by means of light microscopy with polarized light. It has surprisingly been found that the presence of the liquid-crystalline structures in combination with the polymers according to the invention results in stabilization of the formulations with respect to clump formation after thawing. Furthermore, it has surprisingly been found that low stickiness was found. Consequently, the disadvantages caused by dispensing with acrylate-containing polymers were mitigated by the present invention.

Example 11 is a non-inventive example which shows that it is possible to achieve freezing stability at −10° C. with addition of diols, such as butylene glycol, however there is a significant undesirable increase in the stickiness.

Sensory evaluation method of the stickiness for the present emulsions:
1. Division of the inner forearm into two test areas
2. Washing of the corresponding part of the arm (not the entire forearm) and the hands
3. After a waiting time of 3 minutes, 200 μl of an emulsion is applied to the middle of the palm
4. The emulsion is then spread over the test area with the whole palm using circular movements (one circle per second).
5. The emulsion is spread until the product has been absorbed (max. 90 seconds)
6. The ball of the hand is then pressed once onto the test site (area between inside and outside of the forearm) and the attribute stickiness is assessed (0 not sticky, 5 very sticky).

Each formulation was measured 6 times by test subjects.

| | Ex. | Ex. | Ex. | Ex. | Ex. | Ex. | Ex. | Ex. | Ex. | Ex. | Ex. |
|---|---|---|---|---|---|---|---|---|---|---|---|
| Ingredients | 1 | 2 | 3 | 4 | 5 | 6 | 7 | 8 | 9 | 10 | 11 |
| Vegetable Oil | 0.3 | 0.3 | 0.3 | 0.3 | 0.3 | 0.3 | 0.3 | 0.3 | 0.3 | 0.3 | 0.3 |
| Coco-Caprylate/ | 1.1 | 1.1 | 1.1 | 1.1 | 1.1 | 1.1 | 1.1 | 1.1 | 1.1 | 1.1 | 1.1 |
| Caprate | | | | | | | | | | | |
| Simmondsia | 0.1 | 0.1 | 0.1 | 0.1 | 0.1 | 0.1 | 0.1 | 0.1 | 0.1 | 0.1 | 0.1 |
| Chinensis | | | | | | | | | | | |
| Seed Oil | | | | | | | | | | | |
| Prunus | 0.2 | 0.2 | 0.2 | 0.2 | 0.2 | 0.2 | 0.2 | 0.2 | 0.2 | 0.2 | 0.2 |
| Amygdalus | | | | | | | | | | | |
| Dulcis Oil | | | | | | | | | | | |
| Myristyl | 1.5 | 1.5 | 1.5 | 1.5 | 1.5 | 1.5 | 1.5 | 1.5 | 1.5 | 1.5 | 1.5 |
| Myristate | | | | | | | | | | | |
| Hydrogenated | 1 | 1 | 1 | 1 | 1 | 1 | 1 | 1 | 1 | 1 | 1 |
| Coco- | | | | | | | | | | | |
| Glycerides | | | | | | | | | | | |
| Triisostearin | 0.3 | 0.3 | 0.3 | 0.3 | 0.3 | 0.3 | 0.3 | 0.3 | 0.3 | 0.3 | 0.3 |
| Palmitic Acid | 1.1 | 1.1 | 1.1 | 1.1 | 1.1 | 1.1 | 1.1 | 1.1 | 1.1 | 1.1 | 1.1 |
| Stearic Acid | 1.1 | 1.1 | 1.1 | 1.1 | 1.1 | 1.1 | 1.1 | 1.1 | 1.1 | 1.1 | 1.1 |
| Glyceryl | 1.1 | 1.1 | 1.1 | 1.1 | 1.1 | 1.1 | 1.1 | 1.1 | 1.1 | 1.1 | 1.1 |
| Stearate | | | | | | | | | | | |
| Cetearyl | 1.1 | 1.1 | 1.1 | 1.1 | 1.1 | 1.1 | 1.1 | 1.1 | 1.1 | 1.1 | 1.1 |
| Glucoside | | | | | | | | | | | |
| Sorbitan | 0.5 | 0.5 | 0.5 | 0.5 | 0.5 | 0.5 | 0.5 | 0.5 | 0.5 | 0.5 | 0.5 |
| Stearate | | | | | | | | | | | |
| Cetearyl | 0.1 | 0.1 | 0.1 | 0.1 | 0.1 | 0.1 | 0.1 | 0.1 | 0.1 | 0.1 | 0.1 |
| Alcohol | | | | | | | | | | | |
| Disodium | 0.1 | 0.1 | 0.1 | 0.1 | 0.1 | 0.1 | 0.1 | 0.1 | 0.1 | 0.1 | 0.1 |
| Cetearyl | | | | | | | | | | | |
| Sulfosuccinate | | | | | | | | | | | |
| Perfume | 0.2 | 0.2 | 0.2 | 0.2 | 0.2 | 0.2 | 0.2 | 0.2 | 0.2 | 0.2 | 0.2 |
| Glycerol | 4.5 | 4.5 | 4.5 | 4.5 | 4.5 | 4.5 | 4.5 | 4.5 | 4.5 | 4.5 | 7.5 |
| Butylene | | | | | | | | | | | 3 |
| Glycol | | | | | | | | | | | |
| Sodium | q.s. | q.s. | q.s. | q.s. | q.s | q.s. | q.s. | q.s. | q.s. | q.s. | q.s. |
| Hydroxide | | | | | | | | | | | |
| Phenoxyethanol | 0.9 | 0.9 | 0.9 | 0.9 | 0.9 | 0.9 | 0.9 | 0.9 | 0.9 | 0.9 | 0.9 |
| Alcohol Denat. | 3 | 3 | 3 | 3 | 3 | 3 | 3 | 3 | 3 | 3 | 3 |
| Myristyl | 3 | 3 | 3 | 3 | 3 | 3 | 3 | 3 | 3 | 3 | 3 |
| Alcohol | | | | | | | | | | | |
| Hydroxypropyl | | | 0.8 | | 0.8 | | 0.8 | 0.8 | 0.8 | | 0.8 |
| Starch | | | | | | | | | | | |
| Phosphate | | | | | | | | | | | |
| Distarch | | | | | | 0.8 | | | | 0.8 | |
| Phosphate | | | | | | | | | | | |
| Carbomer | 0.3 | | | | | | | | | | |
| Gellan Gum | | | | 0.2 | | | | 0.05 | 0.05 | 0.05 | |
| Xanthan Gum | | | | | 0.05 | | 0.15 | 0 | 0.05 | 0.05 | |
| Aqua | Ad 100 | Ad 100 | Ad 100 | Ad 100 | Ad 100 | Ad 100 | Ad 100 | Ad 100 | Ad 100 | Ad 100 | Ad 100 |
| −10° C., 30 | 80 | 60 | 60 | 65 | 60 | 60 | 65 | 75 | 75 | 75 | 80 |
| d softness/clump | | | | | | | | | | | |
| formation, | | | | | | | | | | | |
| (100% = soft, | | | | | | | | | | | |
| smooth, uniform, | | | | | | | | | | | |
| no clump | | | | | | | | | | | |
| formation) | | | | | | | | | | | |
| Stickiness, | 1.67 | 2.33 | | | | | | | 2.33 | 2.17 | 3.67 |
| average | | | | | | | | | | | |
| (n = 6) | | | | | | | | | | | |
| (1 = not | | | | | | | | | | | |
| sticky, 5 | | | | | | | | | | | |
| very sticky) | | | | | | | | | | | |

The further examples are intended to further illustrate the invention without limiting it.

| Ingredients | Ex. A | Ex. B | Ex. C | Ex. D | Ex. E | Ex. F |
|---|---|---|---|---|---|---|
| Tocopheryl Acetate | 0 | 0.1 | 0 | 0 | 0.1 | 0 |
| Vegetable Oil | 0.2 | 0.5 | 0.3 | 0 | 0 | 0 |
| Helianthus Annuus | 0 | 0 | 0 | 0 | 0.2 | 1 |
| Seed Oil | | | | | | |
| Coco-Caprylate | 0 | 0 | 0.8 | 0 | 0 | 0 |
| Caprylic/Capric | 0.1 | 0 | 0 | 0 | 0.5 | 0 |
| Triglyceride | | | | | | |
| Octyldodecanol | 0 | 0 | 0 | 1 | 0 | 0 |
| Decyl Oleate | 0.1 | 0 | 0 | 0.2 | 0 | 0 |
| Ethylhexyl Cocoate | 0 | 0 | 0.4 | 0 | 0 | 0.1 |
| Dicaprylyl Ether | 0 | 1.5 | 0 | 0 | 0.1 | 0 |
| Coco-Caprylate/Caprate | 1 | 0 | 0 | 0 | 0 | 0.5 |
| Simmondsia Chinensis | 0.1 | 0 | 0 | 0 | 0.2 | 0 |
| Seed Oil | | | | | | |
| Prunus Amygdalus | 0.1 | 0 | 0 | 0 | 0 | 0.2 |
| Dulcis Oil | | | | | | |
| Macadamia Integrifolia | 0 | 0 | 0.1 | 0 | 0.2 | 0 |
| Seed Oil | | | | | | |
| Butyrospermum | 0 | 0 | 0.2 | 0.2 | 0.5 | 0 |
| Parkii Butter | | | | | | |
| Myristyl Myristate | 1.2 | 1.5 | 1 | 2 | 1 | 1.5 |
| Cetyl Palmitate | 0 | 0 | 0.2 | 0 | 0 | 0 |
| Hydrogenated | 0 | 0 | 0.1 | 0 | 0 | 0.2 |
| Rapeseed Oil | | | | | | |
| Cetyl Alcohol | 0.5 | 0.3 | 0 | 0 | 0 | 0 |
| Hydrogenated Coco- | 0.7 | 1.2 | 1.2 | 1.5 | 1 | 2 |
| Glycerides | | | | | | |
| Triisostearin | 0.5 | 0.1 | 0.1 | 0 | 0 | 0 |
| Palmitic Acid | 1 | 1.3 | 1.3 | 0.5 | 1.8 | 1.3 |
| Stearic Acid | 1 | 1.3 | 1.3 | 0.5 | 1.8 | 1.3 |
| Glyceryl Stearate | 1.2 | 1.5 | 1.2 | 0.8 | 0.7 | 1 |
| Cetearyl Glucoside | 1.8 | 0.9 | 2 | 2 | 1.5 | 1 |
| Sorbitan Stearate | 0.8 | 0.4 | 1 | 0.2 | 0.5 | 0.5 |
| Cetearyl Alcohol | 0.1 | 0.1 | 0.3 | 0.5 | 1 | 1 |
| Disodium Cetearyl | 0.1 | 0.1 | 0.1 | 0 | 0 | 0 |
| Sulfosuccinate | | | | | | |
| Perfume | 0.1 | 0.2 | 0.3 | 0.4 | 0.5 | 0 |
| Glycerol | 4.5 | 4.5 | 7 | 10 | 4 | 8 |
| Aqua + Sodium | q.s. | q.s. | q.s. | q.s. | q.s. | q.s. |
| Hydroxide | | | | | | |
| Phenoxyethanol | 0.9 | 0 | 0.7 | 0.9 | 0.8 | 0 |
| Benzyl Alcohol | 0 | 0.5 | 0 | 0 | 0 | 0.2 |
| Ethylhexylglycerin | 0 | 0.3 | 0 | 0.1 | 0 | 0.2 |
| Alcohol Denat. | 3 | 5 | 3.5 | 2 | 4 | 7 |
| Myristyl Alcohol | 3.5 | 4 | 3.5 | 3 | 3 | 5 |
| Hydroxypropyl | 0.5 | 0 | 0.8 | 0.2 | 0 | 0.5 |
| Starch Phosphate | | | | | | |
| Distarch Phosphate | 0 | 2 | 0 | 1 | 0.5 | 0 |
| Sclerotium Gum | 0.2 | 0 | 0 | 0 | 0.5 | 0.1 |
| Gellan Gum | 0.1 | 0.1 | 0.1 | 0.2 | 0.1 | 0.2 |
| Xanthan Gum | 0.1 | 0.1 | 0.1 | 0.1 | 0.1 | 0 |
| Aqua | Ad 100 | Ad 100 | Ad 100 | Ad 100 | Ad 100 | Ad 100 |

1.-15. (canceled)

16. A cosmetic emulsion, wherein the emulsion is acrylate-free and comprises
(a) an emulsifier system which forms a liquid-crystalline network in the emulsion,
(b) Hydroxypropyl Starch Phosphate and/or Distarch Phosphate,
(c) Gellan Gum.

17. The emulsion of claim 16, wherein the emulsion is an oil-in-water emulsion.

18. The emulsion of claim 16, wherein the emulsifier system comprises at least one fatty alcohol glucoside.

19. The emulsion of claim 16, wherein the emulsion comprises from 0.5% to 3.5% by weight of Cetearyl Glucoside, based on a total weight of the emulsion.

20. The emulsion of claim 16, wherein the emulsion comprises from 0.9% to 2.2% by weight of Cetearyl Glucoside, based on a total weight of the emulsion.

21. The emulsion of claim 16, wherein the emulsion comprises a total of from 0.1% to 5.0% by weight of one or more fatty alcohol glucosides, based on a total weight of the emulsion.

22. The emulsion of claim 16, wherein the emulsion comprises a total of from 0.9% to 2.2% by weight of one or more fatty alcohol glucosides, based on a total weight of the emulsion.

23. The emulsion of claim 16, wherein the emulsifier system comprises Sorbitan Stearate.

24. The emulsion of claim 16, wherein the emulsifier system comprises from 0.1% to 1.5% by weight of Sorbitan Stearate, based on a total weight of the emulsion.

25. The emulsion of claim 16, wherein the emulsifier system comprises Glyceryl Stearate.

26. The emulsion of claim 16, wherein the emulsifier system comprises from 0.1% to 1.8% by weight of Glyceryl Stearate, based on a total weight of the emulsion.

27. The emulsion of claim 16, wherein the emulsifier system comprises myristyl alcohol.

28. The emulsion of claim 16, wherein the liquid-crystalline network is present as a liquid-crystalline structure having a globular structure.

29. The emulsion of claim 16, wherein the emulsion comprises from 0.1% to 3.0% by weight of Hydroxypropyl Starch Phosphate and/or Distarch Phosphate, based on a total weight of the emulsion.

30. The emulsion of claim 16, wherein the emulsion comprises from 0.25% to 0.9% by weight of Hydroxypropyl Starch Phosphate and/or Distarch Phosphate, based on a total weight of the emulsion.

31. The emulsion of claim 16, wherein the emulsion comprises from 0.01% to 0.5% by weight of Gellan Gum, based on a total weight of the emulsion.

32. The emulsion of claim 16, wherein the emulsion comprises from 0.04% to 0.2% by weight of Gellan Gum, based on a total weight of the emulsion.

33. The emulsion of claim 16, wherein the emulsion further comprises Xanthan Gum.

34. The emulsion of claim 16, wherein the emulsion comprises less than 0.5% by weight of short-chain diols having from 3 to 5 carbon atoms.

35. The emulsion of claim 16, wherein the emulsion further comprises at least one wax component having a melting point of from 25° C. to 43° C.
